(19) United States
(12) Patent Application Publication
(10) Pub. No.: US 2023/0414370 A1
(43) Pub. Date: Dec. 28, 2023

(54) Implant for Femoral Head Ostectomy

(71) Applicant: Robert Mark Daye, Medina, OH (US)

(72) Inventor: Robert Mark Daye, Medina, OH (US)

(21) Appl. No.: 18/215,507

(22) Filed: Jun. 28, 2023

Related U.S. Application Data
(60) Provisional application No. 63/356,268, filed on Jun. 28, 2022.

(51) Int. Cl. A61F 2/34; A61F 2/36; A61B 17/86

(52) U.S. Cl. CPC A61F 2/34; A61F 2/3609; A61B 17/8615; A61F 2002/365

(57) ABSTRACT

The device is an implant or prosthesis for use in veterinary surgery. The implant or prosthesis is typically spherical and made of silicone, Nylon, Teflon, polyethylene, high density polyethylene (HDPE), ultra high molecular weight polyethylene (UHMWPE) or similar material of appropriate mechanical and physiologically inert characteristics. After a femoral head ostectomy wherein the head of a femur is removed, the prosthesis is inserted into the acetabulum and secured thereto. The prosthesis provides a neutral buffer between the pelvis and the surgically cut surface of the femur.

### RELATED APPLICATIONS

The present application claims priority of U.S. Provisional Patent Application No. 63/356,268 filed on Jun. 28, 2022, the entire disclosure of which is expressly incorporated herein by reference.

### BACKGROUND

### Technical Field

The present disclosure relates to an implant, typically for use in veterinary surgery, such as a femoral head ostectomy (FHO) with an animal, such as, but not limited to, a canine, a feline, or other quadruped animal species, both domestic and exotic.

### RELATED ART

A femoral head ostectomy (FHO) is a veterinary surgical procedural, typically for canines, felines, or other quadruped animal species, with a goal of substantially restoring pain-free mobility to a diseased or damaged hip, by removing the head and neck of the femur.

The normal hip of a canine, as well as of many other mammals, is a ball-and-socket joint with the socket (or acetabulum) formed on the pelvis and the ball formed at the end or head of the femur. That is, the ball is formed as a projection from the long bone located between the hip and the knee thereby providing a ball to rotate within the socket or acetabulum of the hip, thereby allowing the hip to move freely.

When the hip becomes damaged or diseased, such as by hip fractures, hip luxation/dislocation, severe arthritis of the hip or Legg-Perthes disease (avascular necrosis of the femoral head), this mobility can be affected. If the acetabulum and the head of the femur do not fit together properly, this poor fit can influence the degree of movement that the joint can achieve. In addition, this poor joint fit can lead to chronic pain and inflammation.

An FHO involves the surgical removal of the head of the femur thereby restoring mobility to the hip. This removal of the ball from the ball-and-socket configuration results in an empty socket in the hip. The muscles of the leg will typically initially hold the femur in place and, over time, scar tissue will form between the acetabulum and the femur to provide cushioning in what is referred to as a “false joint.” Although this joint is anatomically very different from the natural or normal hip joint, it provides mobility with at least substantially reduced pain in most veterinary patients.

The FHO procedure is primarily recommended for small dogs, such as those under approximately fifty pounds, and cats, especially those at a healthy weight. The false joint that is created in an FHO works very well to support the weight of small animals, but, with exceptions, may be less effective in large-breed dogs. Function is not generally considered normal and maximum effectiveness often requires prolonged recovery and physical rehabilitation.

Active dogs often experience better results with FHO than less-active dogs. The muscle mass that has been built up through activity helps to stabilize the joint, allowing the dog to regain pain-free mobility more quickly than inactive dogs. Inactive dogs have less muscle mass around the joint, making the joint less stable post-operatively and leading to longer recovery times and less rewarding results.

While this procedure is well-suited for its intended purposes, improvements may be sought with respect to femur positioning, effective femur length, hip deviation, natural limb angles and natural gait. Furthermore, grinding and crepitation, which may cause discomfort, because of boney interference, is a concern in such procedures. This may delay or limit the extent of recovery.

### SUMMARY

The present disclosure relates the use of a spherical implant or prosthesis, typically made of silicone, Nylon, Teflon, polyethylene, high density polyethylene (HDPE), ultra high molecular weight polyethylene (UHMWPE) or similar material, which is inserted into the socket or acetabulum of a veterinary patient after a femoral head ostectomy. The implant includes an axial passageway with a first half of the length of the passageway having a diameter which is greater than the diameter of the second half of the length of the passageway thereby resulting in a countersunk configuration. The spherical implant is fixed in position within the acetabulum by a screw that passes through the axial passageway, the head of the screw engaging a washer which is seated on the shelf formed by the reduction in diameter between the first and second halves of the length of the passageway. The screw extends through the second half of the length of passageway and extends into the bone of the acetabulum to be affixed thereto. The opening of the first half of the length of the passageway reduces some of the spherical character of the implant or silicone prosthesis and provides a neutral buffer between the pelvis and the surgically cut (typically flat) surface of the femur. This is expected to provide for a more comfortable, rapid, and substantially more complete recovery.

### BRIEF DESCRIPTION OF THE DRAWINGS

The foregoing features will be apparent from the following Detailed Description, taken in connection with the accompanying drawings, in which:

FIG. 1 is an upper perspective view of a silicone FHO implant;

FIG. 2 is a side view of the apparatus shown in FIG. 1;

FIG. 3 is a cross-sectional view along plane 3-3 of FIG. 2;

FIG. 4 is a lower perspective view of the apparatus of FIG. 1;

FIG. 5 is a bottom view of the apparatus of FIG. 1;

FIG. 6 is a top view of the apparatus of FIG. 1;

FIG. 7 is a cross-sectional view along plane 3-3 of FIG. 2, showing installation hardware;

FIG. 8 is a side view of the apparatus of FIG. 1 implanted in a canine's leg;

FIG. 9 is a view of a diseased hip, FIG. 10 is view thereof with the femoral head removed (post FHO), and FIG. 11 is a view thereof with an implant attached to the acetabulum.

### DETAILED DESCRIPTION

The present disclosure relates a femoral head ostectomy implant 10 and a method for femoral head ostectomy. The implant is typically made from silicone, Nylon, Teflon, polyethylene, high density polyethylene (HDPE), ultra high molecular weight polyethylene (UHMWPE) or similar material. For example, HDPE or any other suitable material can be used to occupy the space between the cut femur and the acetabulum. The implant is attachable to the acetabulum, not the femur. Further, a typical diameter of the implant 10 may be ten millimeters, but it is envisioned that implant 10 would be provided in a wide range of diameters, depending upon the size of the veterinary patient, particularly the size of the acetabulum of the veterinary patient. The implant could be spherical or generally spherical with flat opposing faces.

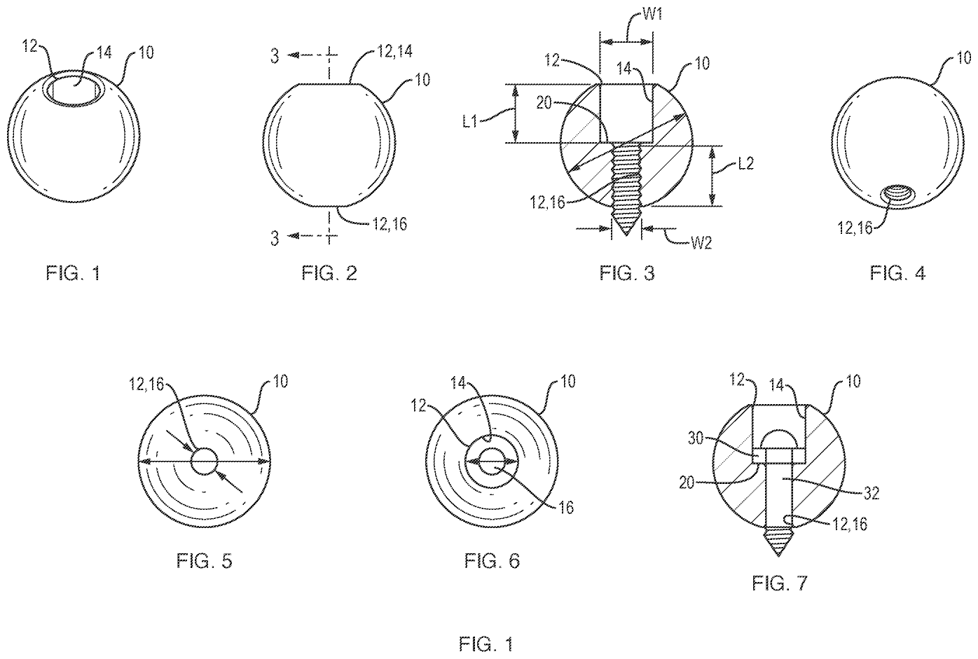

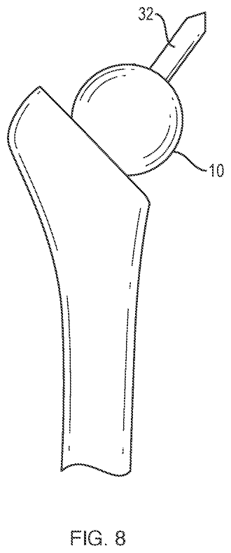

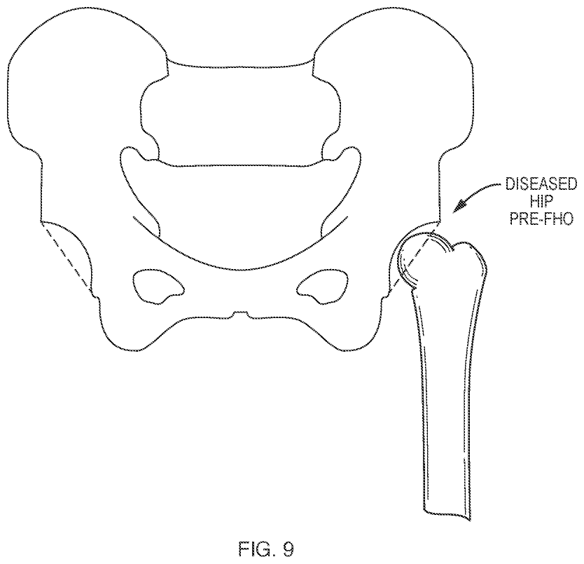

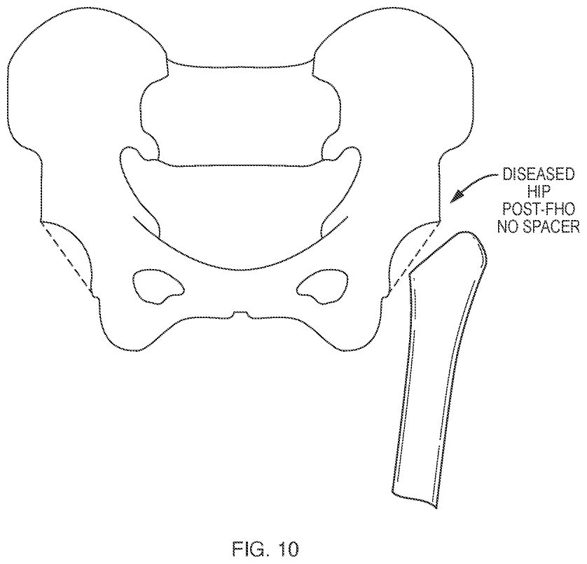

Referring to FIGS. 1-7, passageway 12 is formed through a diameter of the implant 10, with an upper portion 14 of the passageway 12 being larger in diameter than the lower portion 16 of the passageway 12. Cylindrical shelf 20 is formed between the upper and lower portions 14, 16 of the passageway and provides a seat for either a washer 30 (through which a screw 32, as shown in FIG. 7, passes) or a screw 32 in fastening the implant 10 to the veterinary patient.

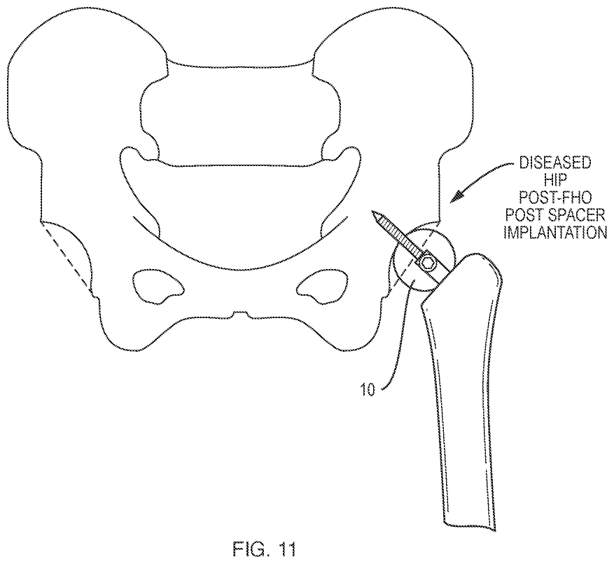

In the installed position, a screw is seated within the passageway 12, extending from lower portion 16 of passageway 12. The screw secures the implant 10 to within the acetabulum of the veterinary patient, with the upper portion of the implant 10 providing a neutral buffer between the pelvis and the surgically cut (typically flat) surface of the femoral neck, as illustrated in FIG. 8. This allows the implant 10 to act as a prosthesis and to rotate with the acetabulum of the veterinary patient.

FIG. 9 is a view of a diseased hip, FIG. 10 is view thereof with the femoral head removed (post FHO), and FIG. 11 is a view thereof with an implant attached to the acetabulum.

Having thus described the system and method in detail, it is to be understood that the foregoing description is not intended to limit the spirit or scope thereof. It will be understood that the embodiments of the present disclosure described herein are merely exemplary and that a person skilled in the art can make any variations and modification without departing from the spirit and scope of the disclosure. All such variations and modifications, including those discussed above, are intended to be included within the scope of the disclosure.

What is claimed is:

1. A veterinary surgical method comprising the steps of:
removing a femoral head from a femur; and
inserting an implant into an acetabulum;
securing the implant to the acetabulum; and
placing the femur against the implant.

2. The method of claim 1 wherein the implant is spherical and the step of securing the implant to the acetabulum comprises inserting a screw into a passageway in the implant and screwing the screw into the acetabulum.

3. A veterinary implant including:
a body;
a passageway through an axis of the spherical body, including a first length of a first diameter and a second length of a second diameter, thereby forming a shelf;
an attachment device extending through the body and into an acetabulum of a veterinary patient to attach the implant to the patient.

4. The implant of claim 3 wherein the body is spherical.

5. The implant of claim 3 wherein the body includes a flat portion.

6. The implant of claim 4 wherein the body comprises silicone.

7. The implant of claim 4 wherein the body comprises high density polyethylene.

8. The implant of claim 4 wherein the attachment device extends through the passageway.

9. The implant of claim 6 wherein the attachment device comprises a screw.

10. The implant of claim 9 wherein the shelf is sized to engage an underside of a head of the screw.

11. The implant of claim 10 further comprising a washer positioned between the underside of the head of the screw and the shelf.
